(19) United States
(12) Patent Application Publication
(10) Pub. No.: US 2018/0106777 A1
(43) Pub. Date: Apr. 19, 2018

(54) APPLICATION OF TIME DERIVATIVE OF DISTRIBUTED TEMPERATURE SURVEY (DTS) IN IDENTIFYING CEMENT CURING TIME AND CEMENT TOP

(71) Applicant: Halliburton Energy Services, Inc., Houston, TX (US)

(72) Inventors: Hongyan Duan, Houston, TX (US); Mikko Jaaskelainen, Katy, TX (US)

(21) Appl. No.: 15/567,850

(22) PCT Filed: Jun. 15, 2015
(86) PCT No.: PCT/US2015/035808

§ 371 Date: Oct. 19, 2017

(51) Int. Cl. G01N 33/38; G01K 11/32; G01H 9/00; E21B 47/06

(52) U.S. Cl. CPC G01N 33/383; E21B 47/065; G01H 9/004; G01K 11/32

(57) ABSTRACT

A method is presented for using the time derivative of distributed temperature sensing data to monitor and analyze cement critical temperature Time derivative of DTS in depth and time scale changes during the cementing process in subsurface wells.

### BACKGROUND

This disclosure relates generally to temperature sensing, and more particularly, to the use of new methodologies for interpreting distributed temperature sensing information.

Fiber optic Distributed Temperature Sensing (DTS) systems were developed in the 1980s to replace thermocouple and thermistor based temperature measurement systems. DTS technology is often based on Optical Time-Domain Reflectometry (OTDR) and utilizes techniques originally derived from telecommunications cable testing. Today DTS provides a cost-effective way of obtaining hundreds, or even thousands, of highly accurate, high-resolution temperature measurements, DTS systems today find widespread acceptance in industries such as oil and gas, electrical power, and process control.

DTS technology has been applied in numerous applications in oil and gas exploration, for example hydraulic fracturing, production, and cementing among others. The collected data demonstrates the temperature profiles as a function of depth and of time during a downhole sequence. The quality of the data is critical for interpreting various fluid movements.

The underlying principle involved in DTS-based measurements is the detection of spontaneous Raman back-scattering. A DTS system launches a primary laser pulse that gives rise to two back-scattered spectral components. A Stokes component that has a lower frequency and higher wavelength content than the launched laser pulse, and an anti-Stokes component that has a higher frequency and lower wavelength than the launched laser pulse. The anti-Stokes signal is usually an order of magnitude weaker than the Stokes signal (at room temperature) and it is temperature sensitive, whereas the Stokes signal is almost entirely temperature independent. Thus, the ratio of these two signals can be used to determine the temperature of the optical fiber at a particular point. The time of flight between the launch of the primary laser pulse and the detection of the back-scattered signal may be used to calculate the spatial location of the scattering event within the fiber.

DTS technology has been applied to cement monitoring in down-hole wells. DTS data has been used to better monitor the cement injection process where the location of the un-cured cement can be monitored over time as a moving temperature event as cement is pumped into the well, and to identify the depths where cement curing occurs in subsurface wells. is Successful primary cementing operations result in a cement sheath to bond and support casing and provide zonal isolation. Good zonal isolation helps prevent the loss of production, control inter-zonal flow and/or flow to the surface, reduce water production and improve confinement of stimulation treatments. The location of the cement, and curing times are critical in evaluating a cement job.

Cement curing is a chemical reaction that releases energy. The released heat causes a temperature increase that is faster than the geothermal heating. The quest for deeper insights into the data for guiding understanding of what is happening during the curing process is a need.

Despite the usefulness of normal DTS data in interpreting what is happening in cementing operations there is a need for even better monitoring. A long felt need is for better capturing the temperature changes occurring during the operation to improve the quality of the final cement job. A useful methodology for capturing these changes and displaying them for operator analysis will be presented in this disclosure.

### BRIEF DESCRIPTION OF THE DRAWINGS

FIG. 1 illustrates sample DTS data in the depth and time scale during a cementing process.

FIG. 2 illustrates the same DTS data displayed as the time derivative in the depth and time scale.

FIG. 3 illustrates the data matrices representing the DTS data for representing the time derivative display.

FIG. 4 illustrates a workflow for generating the data analysis for the identification.

### DETAILED DESCRIPTION

In the following detailed description, reference is made to accompanying drawings that illustrate embodiments of the present disclosure. These embodiments are described in sufficient detail to enable a person of ordinary skill in the art to practice the disclosure without undue experimentation. It should be understood, however, that the embodiments and examples described herein are given by way of illustration only, and not by way of limitation. Various substitutions, modifications, additions, and rearrangements may be made without departing from the spirit of the present disclosure. Therefore, the description that follows is not to be taken in a limited sense, and the scope of the present disclosure will be is defined only by the final claims.

DTS technology has been applied to cement monitoring in down-hole wells. DTS data can be used to better monitor the cement injection process where the location of the un-cured cement can be monitored over time as a moving temperature event as cement is pumped into the well. A down-hole completion require in most cases that the wellbore above a producing interval is cemented to prevent migration of hydrocarbons to the surface and/or migration of hydrocarbons to zones where hydrocarbons may e.g. contaminate fresh water reservoirs. It is also desirable to monitor the location of different cement interfaces if multiple types of cement is used for various reasons like e.g. different reservoir layers having different properties and cement is chosen to match these properties.

The quality of the cement job is critical and it is desirable to monitor relevant important properties to allow proper evaluation of a cement job. Cement can be designed to have custom properties like curing at certain rates under a given set of conditions (e.g. temperature, pressure, chemical environment) to achieve desired properties. Custom chemistry allows optimization of cement properties like the ability to bond to different materials like reservoir rock and metal casing, thermal expansion, mechanical support and fracture properties when a well is perforated using shaped charges. These cement properties are important when the well is being fractured and during the life of the well to make sure that good zonal isolation is achieved to e.g. avoid cross flow between producing zones and allow proper placement of treatment chemicals.

It is therefore desirable to measure the downhole temperature and the rate at which the cement cures at different locations. This data can be used to evaluate the effectiveness of a cement job and to make sure that the cement is fully cured before commencement of other down-hole operations. Rig-time is expensive and operators want to keep the down-time of the rig to a minimum but it is critical to know that the cement has properly cured before starting down-hole operations after a cement job.

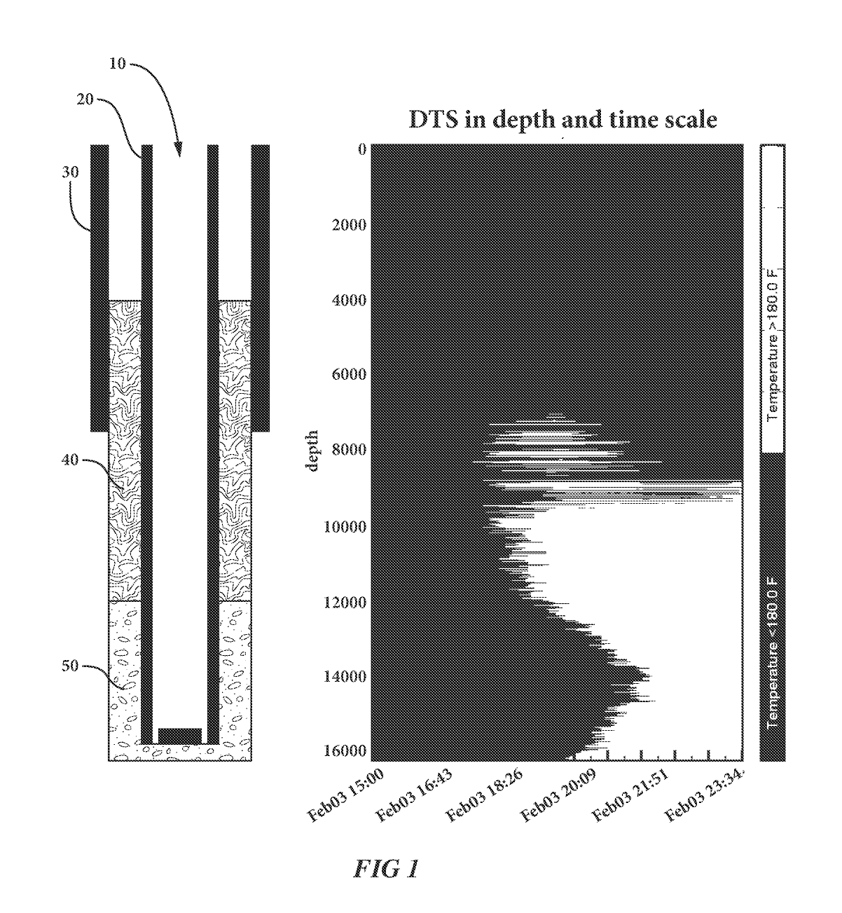

Referring first to FIG. 1, distributed temperature data is displayed in the depth (y-axis) and time (x-axis) scale obtained by a commercial DTS system during the cementing process. A wellbore diagram is exhibited on the left to show completion information. The diagram shows a wellbore 10 defined by a production casing 20 enclosed by a surface casing 30 with cements 40 and 50 that have been pumped to fill the annulus between the casings. Two different types of cements were injected in sequence and the boundary 60 is shown. After all planned cement segments have been pumped, a plug is inserted and water is pumped into the casing to push the plug and seal the plug at the bottom of the casing. This moves all the cement from the inside of the casing and up the annular space. From the DTS plot, it can be roughly interpreted that the earliest cement curing occurs at about 7000 feet depth and the latest cement curing occurs at about 14,000 feet. But duration of the curing time can not be identified from the plot. The DTS data also indicates that no obvious heating is shown above 7000 feet.

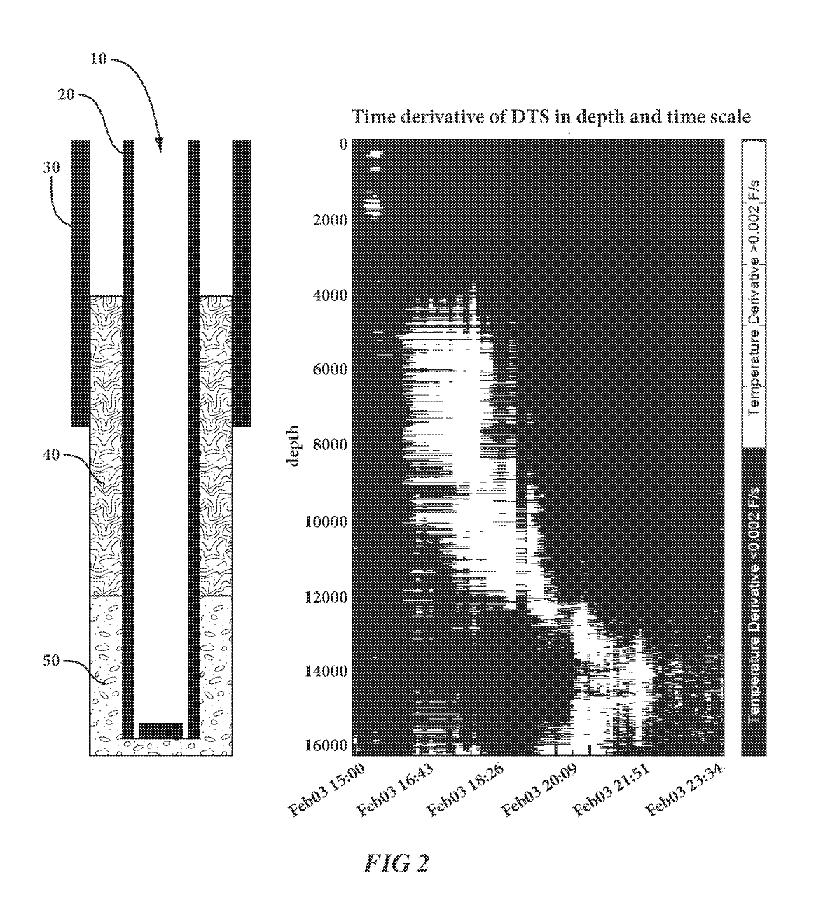

Turning to FIG. 2, with the exact same wellbore diagram the same DTS data is now displayed as the time derivative of the DTS data in the depth and time scale. It should be noted that the data of FIGS. 1 and 2 will normally be displayed in full color to show temperature changes. Color presentations cannot be used in patent applications so this data is being shown in a black/white scale that still shows the significant improvement in information available using derivative data to show the various boundaries during the cement curing process.

Cement pumped down from the surface normally has a different temperature than the formation, and this temperature difference can be observed with a DTS system. The pumping operation is stopped once the cement has reached the appropriate depth, and the DTS data can show the location of the cement as it is pumped down by monitoring the temperature over time. Cement in place starts to increase in temperature due to the geothermal heating, followed by an additional increase in temperature due to the heat generated during the cement curing. From the derivative of DTS plot in FIG. 2, it is easily seen that cement curing stands out as a higher (white) value zone in the plot. Geothermal heating however shows as mix between white and a lower (dark) value before and after the curing. Curing time can be therefore observed as about 2.5 hours for example, as the white band across at the depth of 10,000 feet. That information is not available in FIG. 1. In addition, the cement top can be accurately identified from the derivative map to be near 4000 feet, rather than the 7000 feet shown in FIG. 1 and the boundary between two different types of cement is exhibited as the break at about 12,200 feet and a time of about 20:05 on February 3.

After cement is injected, geothermal heating takes effect immediately. At a certain point after that, cement starts to cure in different rate at different depths due to the shear differentials. In a standard DTS plot, it is difficult to separate the cement curing from the geothermal heating. Therefore the quality of cementing in depths is not easily addressed. The time derivative of DTS is able to capture the temperature increase caused by cement curing and shows geothermal heating as different color tones in the map, or as darker vs lighter in a gray scale rendition, or as black/white images.

This method can be described as using the time derivative of distributed temperature sensing data to monitor cement critical temperature changes during the cementing process in subsurface wells including at least: providing a fiber optic based distributed temperature sensing measurement system through the region to be cemented; gathering the temperatures of the cement from the distributed temperature sensing system as a function of the depth in the subsurface well and as a function of the elapsed time; calculating from the gathered data the time derivative of the temperature changes as a function of depth in the subsurface well and of the elapsed time; displaying the time derivative data for analysis of the cementing process by operators.

Furthermore the time derivative data can be presented in a number of ways. In one embodiment the actual numerical values of the time derivative data are recorded and printed or displayed. In another embodiment the time derivative data can be displayed in colors as a function of depth and time on a display monitor. In another embodiment the time derivative data can be displayed in gray scale as a function of depth and time on a display monitor.

Distributed Acoustic Sensing (DAS) systems may also be used to monitor cement curing and cement top location, and may enhance the data interpretation based on DTS derivative. Acoustic energy may travel at different velocities in the annulus if it is filled with air or liquid or cement, and various frequencies may attenuate differently in the various environments. Careful investigation of the acoustic data versus depth may be used with the DTS derivative data to identify cement location. Similarly, thermal variations may change the effective fiber length due to thermal expansion and may cause changes in optical path length that may be used to measure slow thermal changes. The optical path length may therefore increase due to thermal expansion of the optical fiber as the cement cures, and similarly the optical path length may decrease as the cement has stopped curing and cools down to the temperature of the rock formation. This can be used together with the DTS derivative method to identify cement curing time over time and depth.

Generation of Derivative DTS Data

The disclosure herein anticipates any mathematically correct manner of generating the derivative data. The example embodiments for calculating the time derivative are explained below.

Derivative data from DTS data can be generated by feeding the numerical data of temperature as a function of depth and time into a matrix and then computationally moving through all of the matrix data points to calculate derivative values for each matrix element. This can be done as either depth derivatives or as time derivatives. These derivative values can then be presented as a matrix of numbers, or, more usefully can be presented as color images in which the various colors represent different values of the derivatives. As discussed earlier, they are presented herein as black/white scale images which show important features that are not evident in the presentation of the conventional DTS data alone.

Time Derivative of DTS:

In this example the computation language MatLab is used to compute regular DTS data into a time derivative of DTS. And the result is also plotted by MatLab in a depth-time scale.

For the DTS measurement, temperature is function of depth and time:

T=T(depth, time) (3)

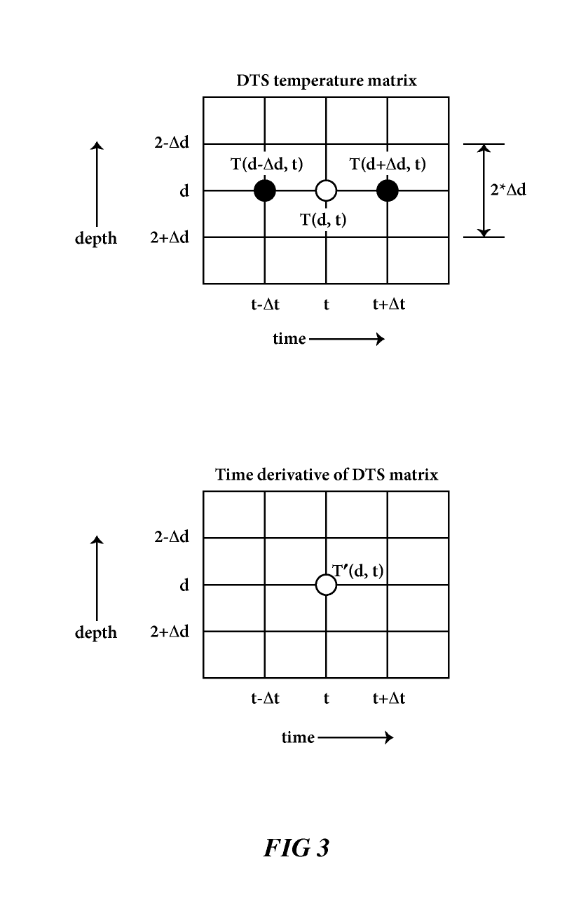

Data is loaded into MatLab and stored as a matrix. See the first matrix of FIG. 3.

The time derivative of DTS, also called DTS time gradient, is computed as:

T̂′(d,t)=(T(d,t+Δt)−T(d,t−Δt))/(2*Δt) (4)

The time derivative at any depth and time step is calculated by subtracting the temperature at its previous time step from the one at its next time step and result is divided by the time interval between these two steps.

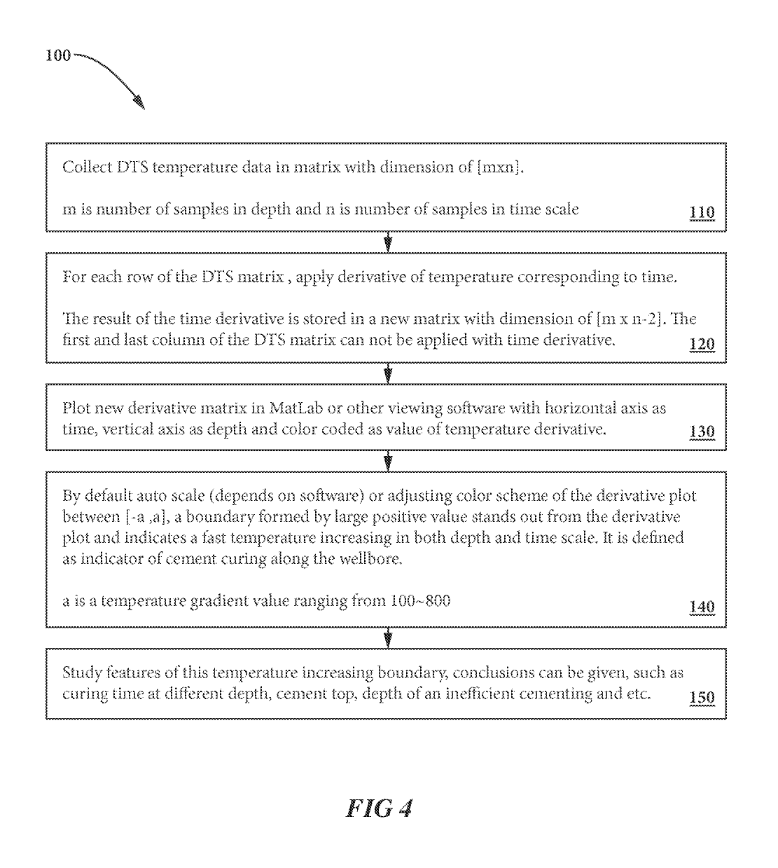

The structure of the derivative matrix is shown as the second matrix in FIG. 4:

Both DTS and DTS derivative matrix can be plotted as a depth-time 2D color map by MatLab function pcolor(d,t,T) or pcolor(d,t,T′). Input parameters d and t are depth and time vectors. Input T or T′ is a 2D matrix with number of rows as d and number of columns as t.

The method can be described alternatively with the process 100 as in FIG. 4. In the first step 110 a DTS system is used to collect the distributed temperature data into a DTS matrix with dimensions of [m×n], where m is the number of samples taken in the depth scale and n is the number of samples taken in time scale. In step 120, a de-noising algorithm is applied on the saved DTS matrix before the derivative application, and the data is averaged in time and depth windows and size of the window depends on sampling rate and data quality. After the de-noising process, a derivative calculation is performed for each column of the DTS matrix, and the derivative of temperature with respect to time is calculated. The result of this derivative is stored in a new matrix with dimension [m×n−2]. The first and last column of the DTS matrix cannot be applied with the time derivative. The developing time derivative matrix is shown in FIG. 3. In the step 130 any viewing software such as MatLab can be used to plot the derivative matrix with time as the horizontal axis and depth as the vertical axis. If color display is operable the color can be coded as a value of temperature derivative. Most of the plotting software offers a reasonable auto scale enough to show most of features from a derivative plot. In case there is an extreme value caused by artifacts, such as a large temperature jump (positive or negative), the user can then adjust (step 140) the color scheme of the derivative plot until a boundary formed by large positive value stands out at expected cementing depths. For cement curing, this value is not more than 0.01 degree F./second across all of the time scale in the derivative plot of FIG. 2. The observed boundary then can indicate a fast temperature increase in time-depth plot. Typical geothermal warming is in speed of 0.0001-0.001 degree F./second depends on depth of well. It shows as a background color in a derivative plot. Temperature increase caused by cement curing normally ranges from 0.002 to 0.006 degree F./second at most of the depths, 2 to 6 times higher then the maximum geothermal warming. It is a viable indicator of cement curing along a wellbore. Comparison of FIGS. 1 and 2 show that such information is simply not available in a standard DTS display. Finally, in the last (150) step the analyst can visually examine the features of this temperature increase boundary and reach conclusions regarding curing time at different depth, where the cement top is, the depth of an inefficient cement job, etc.

By default, MatLab uses a Blue-Red color scheme represent the value of the temperature or value of the derivative. In the DTS plot, blue represents a low temperature while red represents a high temperature. In DTS time derivative (DTS time gradient) plot, blue represents a temperature decrease along the time. Red represents a temperature increase along the time. A large value in red (darker) zone indicates a large temperature increase per second. Large negative value in blue zone indicates a large temperature drop per second. Again because color cannot be used in patent applications these are presented as black/white scale images which still show the new possibilities of data presentation possible by the use of displayed color data.

The resulting time derivative temperature data as a function of depth and time can be presented in a number of ways. In one example the actual is numerical values can be stored for later retrieval and then either displayed on a monitor or printed for study. In another example the resulting time derivative of temperature can be displayed as different colors on a color display for better understanding and interpretation. In yet another example that same data can be displayed in black/white scale as shown in FIGS. 1 and 2. The same data can also be displayed in gray scale.

This methodology offers a more accurate monitoring tool than conventional distributed temperature sensing in the monitoring and analysis of the cementing process in subsurface wells.

Although certain embodiments and their advantages have been described herein in detail, it should be understood that various changes, substitutions and alterations could be made without departing from the coverage as defined by the appended claims. Moreover, the potential applications of the disclosed techniques is not intended to be limited to the particular embodiments of the processes, machines, manufactures, means, methods and steps described herein. As a person of ordinary skill in the art will readily appreciate from this disclosure, other processes, machines, manufactures, means, methods, or steps, presently existing or later to be developed that perform substantially the same function or achieve substantially the same result as the corresponding embodiments described herein may be utilized. Accordingly, the appended claims are intended to include within their scope such processes, machines, manufactures, means, methods or steps.

1. A method for using the time derivative of distributed temperature sensing data to monitor cement critical temperature changes during the cementing process in subsurface wells comprising:
a. providing a fiber optic based distributed temperature sensing measurement system through the region to be cemented;
b. gathering the temperatures of the cement from the distributed temperature sensing system as a function of the depth in the subsurface well and as a function of the elapsed time;
c. calculating from the gathered data the time derivative of the temperature changes as a function of depth in the subsurface well and of the elapsed time;
d. displaying the time derivative data for monitoring of the cementing process.

2. The method for using the time derivative of distributed temperature sensing data to monitor cement critical temperature changes during the cementing process in subsurface wells of claim 1 wherein the numerical values of the time derivative data are recorded and printed or displayed.

3. The method for using the time derivative of distributed temperature sensing data to monitor cement critical temperature changes during the cementing process in subsurface wells of claim 1 wherein the time derivative data is displayed in colors as a function of depth and time on a display monitor.

4. The method for using the time derivative of distributed temperature sensing data to monitor cement critical temperature changes during the cementing process in subsurface wells of claim 1 wherein the time derivative data is displayed in black/white scale as a function of depth and time on a display monitor.

5. The method for using the time derivative of distributed temperature sensing data to monitor cement critical temperature changes during the cementing process in subsurface wells of claim 1 wherein the time derivative data is displayed in gray scale as a function of depth and time on a display monitor.

6. The method for using the time derivative of distributed temperature sensing data to monitor cement critical temperature changes during the cementing process in subsurface wells of claim 1 further comprising:
a. providing a fiber optic based distributed acoustic sensing measurement system through the production region;
b. gathering the acoustic measurements of the cement from the distributed acoustic sensing system as a function of the depth in the subsurface well and as a function of the elapsed time;
c. displaying the acoustic data for analysis of the cementing process by operators;
d. using the distributed acoustic data in conjunction with the time derivative data to further refine cement location and curing times.

7. A method for using the time derivative of distributed temperature sensing data to monitor cement critical temperature changes during the cementing process in subsurface wells comprising:
a. providing a fiber optic based distributed temperature sensing measurement system through a production region;
b. gathering the temperatures through the production region as a function of the depth in the subsurface well and as a function of the elapsed time;
c. assembling the data into a DTS matrix of [m×n] wherein m is the number of samples collected in the depth scale and n is the number of samples collected in the time scale;
d. for each row of the DTS matrix calculating a derivative of the temperature as a function of time and storing it in a new matrix with dimensions [m−2×n];
e. displaying the derivative matrix with one axis as time and another axis as depth and color coding the value of the temperature derivative;
f. adjusting the color scheme until a boundary is found through the production time period, indicating a fast temperature increase in both the temperature and time scale, indicating cement curing along the wellbore.

8. The method for using the time derivative of distributed temperature sensing data to monitor cement critical temperature changes during the cementing process in subsurface wells of claim 7 wherein the time derivative data is displayed in colors as a function of depth and time on a display monitor.

9. The method for using the time derivative of distributed temperature sensing data to monitor cement critical temperature changes during the cementing process in subsurface wells of claim 7 wherein the calculated display of the derivative matrix is displayed in gray scale.

10. The method for using the time derivative of distributed temperature sensing data to monitor cement critical temperature changes during the cementing process in subsurface wells of claim 7 wherein the calculated display of the derivative matrix is displayed in black and white.

11. The method for using the time derivative of distributed temperature sensing data to monitor cement critical temperature changes during the cementing process in subsurface wells of claim 7 wherein the calculated numerical values of the derivative matrix are recorded and printed or displayed.

12. The method for using the time derivative of distributed temperature sensing data to monitor cement critical temperature changes during the cementing process in subsurface wells of claim 7 further comprising:
a. providing a fiber optic based distributed acoustic sensing measurement system through the production region;
b. gathering the acoustic measurements of the cement from the distributed acoustic sensing system as a function of the depth in the subsurface well and as a function of the elapsed time;
c. displaying the acoustic data for analysis of the cementing process by operators;
d. using the distributed acoustic data in conjunction with the time derivative data to further refine cement location and curing times.
